(19) United States
(12) Patent Application Publication
(10) Pub. No.: US 2026/0259152 A1
(43) Pub. Date: Sep. 3, 2026

(54) CT Scanner

(71) Applicant: Istituto Nazionale Di Fisica Nucleare, Frascati (RM) (IT)

(72) Inventor: Antonio Sarno, Paduli (IT)

(21) Appl. No.: 18/873,040

(22) PCT Filed: Jun. 10, 2023
(86) PCT No.: PCT/IB2023/055989

§ 371 Date: Dec. 9, 2024

(30) Foreign Application Priority Data
Jun. 10, 2022 (IT) 102022000012341

(51) Int. Cl. G01N 23/046

(52) U.S. Cl. CPC G01N 23/046; G01N 2223/1016; G01N 2223/316; G01N 2223/3303; G01N 2223/419

(57) ABSTRACT

Scanning method for making a computed tomography CT includes a source arranged to generate cone beam x-ray radiation, a detector arranged so that the radiation generates a primary footprint completely contained on a surface of the detector and a processor configured to generate a digital image corresponding to a signal generated by the detector when struck by the radiation and configured to reconstruct a three-dimensional attenuation map of a volume containing a sample under investigation, the method including a first step of making the source perform a first rotating movement around an axis of rotation and a second step of making the source perform a second alternating movement, parallel to the axis of rotation so as to identify an approximately sinusoidal motion on a cylindrical or hemicylindrical surface coaxial with the axis of rotation.

### FIELD OF THE INVENTION

The present invention relates to a CT computed tomography scanner.

### STATE OF THE ART

As known, computed tomography with conical x-ray beams called “cone-beam computed tomography” (CBCT) allows the reconstruction of the image of the internal structure of limbs, tissues, organs or objects starting from various radiographic views and making use of a reconstruction software. The CBCT allows the reconstruction of the three-dimensional map of the attenuation coefficients of the materials present in the investigation field. CBCT uses an x-ray generator/tube, a digital detector for the detection of x-rays and a computerized system for acquiring various projections of the sample placed in the field of view of the source.

By “projection” we mean a digital image given by the conversion of the signals generated by the footprint of the x-ray beam on the detector. Generally, processing means are arranged for controlling the source and for generating the digital image from the electrical signals generated by the detector. The “projection”, in the context of this description, has a different meaning from the concept of “geometric footprint” which has a known meaning in itself. The different projections are obtained by varying the position of the tube-detector pair, integral in their movements, and the sample, which is always placed between the first two. Conventionally, the different projection are acquired for various rotation angles of the source/detector pair around a fixed axis called “isocenter” in the proximity of which the sample is placed. A similar result is obtained by keeping the source/detector fixed in space and rotating the sample placed between the two. Subsequently, a software that executes an appropriate algorithm allows the reconstruction of the three-dimensional image of the object or the map in the three-dimensional volume of the linear attenuation coefficients of the materials constituting the sample contained in the volume. CBCT is now widely used in medical diagnostics, particularly in the field of dentomaxillofacial radiology, imaging of the musculoskeletal system, angiography, interventional radiology, planning of radiotherapy treatment plans and in innovative techniques for three-dimensional diagnosis of breast cancer. CBCT also finds its uses in the industrial field for quality checks and metrology of manufactured articles, as well as in the field of safety for content verification. Similarly, the detector can be fixed and the source alone rotate around the isocenter by an angle of less than 180 degrees. In this case we speak of digital tomosynthesis (DT) or digital breast tomosynthesis (DBT) in case this is the irradiated organ. Despite its wide use, mainly due to the high ratio between effectiveness and cost, CBCT presents a significant problem that negatively affects the quality of the reconstructed 3D image and consists in the abundance of scattered radiation or Scatter that reaches the detector.

Scatter radiation is the cause of artifacts known as “cupping artifacts” or “shading artifacts” which appear in the axial slices of the reconstructed three-dimensional image as a distortion of the low-frequency signal. For example, in cupping artifacts, the signal in the reconstructed axial slice has an increasing trend in the radial direction. Furthermore, the presence and detection of scatter radiation increases high-frequency noise in the reconstructed three-dimensional images, reducing the visibility of small details and lesions and the visibility of low-contrast inclusions. The volume for which the three-dimensional attenuation map is reconstructed is indicated with the symbol FOV. The sample under investigation is located within the FOV.

The characteristics contained in the present paragraph form an integral part of the detailed description of the present invention.

### SUMMARY OF THE INVENTION

The purpose of the present invention is to reduce the effect of scatter and cone artifacts, obtaining a higher quality of the reconstructed image.

The basic idea of the present invention is to acquire only the primary footprint of the x-radiation impacting the detector. This result can be obtained in two ways, by limiting the axial extension of the detector, or by windowing the primary footprint, in the sense of eliminating the pixels that fall outside the primary footprint generated by the source on the detector.

Evidently, this result is obtainable when only an axial fraction of the FOV is illuminated, in the sense that the reconstructed volume is completely irradiated in the axial direction only with at least two phases of illumination by the source.

For this purpose, the source performs an alternating movement in the axial direction, which is combined with a rotating movement, so as to completely irradiate the axial extension of the volume being reconstructed. In other words, the volume has axial dimensions at least twice as large as the axial extension of the primary footprint of the source on the lateral surface of the volume being reconstructed.

An approximately sinusoidal motion is therefore generated on a cylindrical or semi-cylindrical surface, coaxial with the axis around which the source is arranged to rotate.

This rotation motion of the source is around the rotation axis, according to which the source remains constantly facing the rotation axis exactly like the lunar movement around the earth.

According to the invention, processing means are suitably configured to control the movement of the source according to the combination of movements described above, to generate the digital image corresponding to the electrical signals generated by the detector and are preferably also configured to generate a three-dimensional reconstruction of the FOV. However, the three-dimensional reconstruction can also be generated by second processing means, such as a computer, operatively interfaced with the first processing means. According to a first preferred variant of the invention, the detector and the sample are fixed, while the radiation source performs a tilting movement with respect to an axis of rotation, approximately barycentric with respect to the sample, and simultaneously performs an alternating movement parallel to the axis approximately barycentric of the sample. According to a second preferred variant of the invention, the detector and the source are integral in rotation with each other, tilting around an axis of rotation approximately barycentric with respect to the sample. At least the source performs an alternating motion parallel to the approximately barycentric axis of the sample. Preferably, both the source and the detector perform this reciprocating motion synchronously and concordantly with each other.

Equivalently, the detector and the source are fixed, while the sample is induced to rotate around its own axis of rotation, approximately barycentric, and intermediate between the source and the detector, while the source and preferably also the detector moves/move with an alternating motion parallel to the axis of rotation.

The dependent claims describe preferred variants of the invention, forming an integral part of the present description.

### BRIEF DESCRIPTION OF THE FIGURES

Further objects and advantages of the present invention will become clear from the detailed description which follows of an embodiment thereof (and of its variants) and from the attached drawings given for purely explanatory and non-limiting purposes, in which

FIG. 1 represents a perspective view of a schematization of the operating principle of the CT scanner object of the present invention;

FIGS. 2a and 2b show respective sections of the diagram of FIG. 1 along two mutually perpendicular planes;

FIG. 2bis shows a variant of the invention with respect to the section of FIG. 2b;

FIG. 3 shows a perspective view of a further schematic representation of the present invention according to a first variant, whereas FIG. 4 shows a similar perspective representation according to a second variant of the invention;

FIG. 5 shows a perspective view of a variant of the invention comprising an external rigid casing and therefore applicable to all the variants shown in the previous figures.

The same reference numbers and letters in the figures identify the same elements or components or functions.

It should also be noted that the terms “first”, “second”, “third”, “superior”, “inferior” and the like may be used herein to distinguish various elements. These terms do not imply a spatial, sequential, or hierarchical order for the modified items unless specifically indicated or inferred from the text.

The elements and characteristics illustrated in the various preferred embodiments, including the drawings, can be combined with each other without however departing from the scope of protection of the present application as described below.

### DETAILED DESCRIPTION

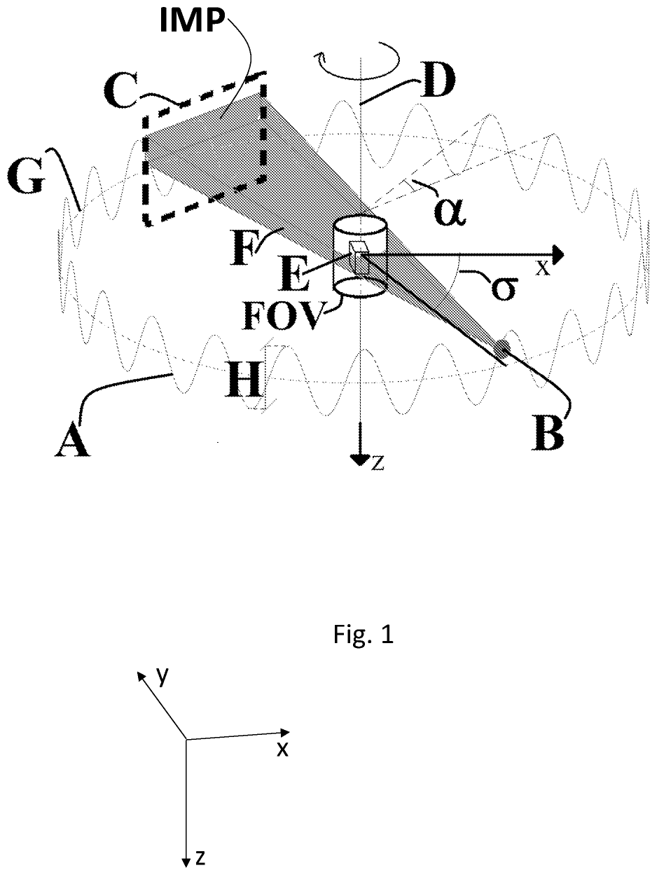

With reference to FIG. 1, the scanner object of the present invention comprises a cone beam x-ray source B and a digital detector C. Preferably, they are mutually supportive.

Detector C is arranged so as to be affected by the radiation generated by the source which passes through sample E. More precisely, the primary footprint is always contained in the detector surface. The x-ray beam is collimated in the x-y plane perpendicular to the axis of rotation and defines the diameter of the FOV (f). This collimation can be coincident with the x-y plane extension of the detector. Examples of scanner operation are described below.

The FOV symbol indicates the volume for which the three-dimensional attenuation map or three-dimensional image is reconstructed. This volume contains a sample E more or less tightly.

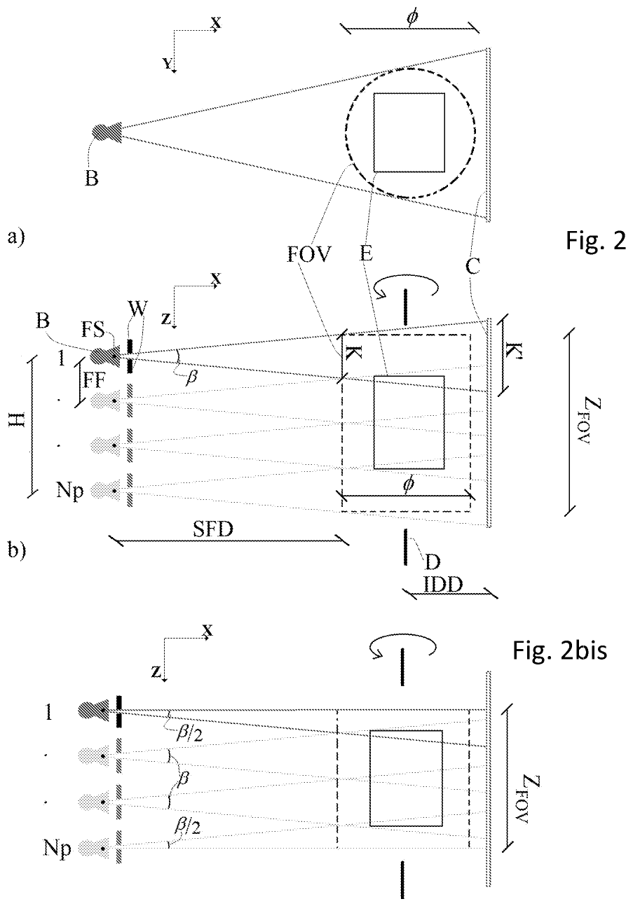

From the comparison of FIGS. 2a and 2b, the sample E is represented in a parallelepiped shape, while the FOV always has a cylindrical or semi-cylindrical shape in which the sample E is inscribed. Obviously, the sample can have any shape.

The D axis is an axis of rotation at least for the source and more preferably for the source/detector pair or for a sample E under investigation CT. When the source/detector pair are arranged to rotate about the axis of rotation D, the sample E remains stationary during the investigation. Since a reciprocal movement must be achieved, this means that at least the source rotates around the rotation axis D while remaining constantly oriented towards the rotation axis D to illuminate the detector. Alternatively, the source and the detector are constantly fixed, while the sample E rotates around the axis of rotation D. Preferably, sample E is arranged in the FOV so that axis D is approximately barycentric.

By convention it is assumed that the axis D of rotation and the axis z of the reference system shown in the figures coincide.

Therefore, the volume has an axial extension, in the direction indicated by the rotation axis D.

It is worth noting that the D axis of rotation is not necessarily equidistant from the source and the detector. This aspect is clear from the comparison of the diagram of FIG. 1 in which the rotation D axis is equidistant with respect to the source and the detector, and FIG. 2b in which it can be seen that the rotation D axis, approximately barycentric for the sample E, is closer to the detector than to the source. The distance between the detector and the rotation axis D is indicated with IDD.

According to the present invention, during the reciprocal rotation between the source and the sample, an alternating axial movement is also generated, i.e. parallel to the D axis, so that the source B traces an approximately sinusoidal trajectory on the cylindrical or hemicylindrical surface of the FOV in the composition of the motion alternating axial and rotational about the axis of rotation D: see FIG. 1. The axial movement of the source, parallel to the axis D, has amplitude H with a medial point Zs, so that the interval of axial oscillation has a predetermined amplitude [Zs+H/2; Zs−H/2]. The amplitude value can be suitably varied. During the rotation about the rotation axis D, a plurality of reciprocating axial movements is performed. “Oscillation cycle” Oc is defined as a movement between two consecutive end positions in Zs+H/2 or in Zs−H/2. The oscillation cycle is associated with a period α between the two consecutive end positions, shown in FIG. 1.

Scanning can be done with rotations smaller or larger than a full rotation of 360 degree.

With reference to FIG. 2b, it can be seen that the source B assumes a number Np=4 axial positions, so as to irradiate the surface of the FOV in the period α. In other words, for each oscillation cycle, i.e. for each oscillation period, Np acquisitions are made with Np>=2. Evidently each acquisition identifies a 2D footprint acquired by the detector.

The angular aperture of the beam of radiation x is indicated by B along the axial direction identified by the rotation axis D.

The period α, the aperture β and the number of acquisitions Np are chosen so that the lateral surface of the FOV is scanned with sufficient continuity both in the axial and annular directions.

Preferably, a collimator W allows to adapt the beam aperture β in such a way that only an axial fraction of the FOV is illuminated at a time and the illumination of the entire axial extension of the FOV surface is achieved thanks to the aforementioned alternating movement parallel to the D axis. If Np=4, it means that 4 projections are needed on the lateral surface of the FOV to fully illuminate it in the axial direction.

To obtain correct illumination it is advisable that the different footprints on the surface of the FOV are contiguous and without overlapping in a single period of oscillation, described below. In other words, it is desired that the x-radiation footprints on the FOV surface are axially contiguous to each other.

The axial positions Np of the source correspond to the same number of geometric footprints on the FOV with amplitudeK=Z_{FOV}/Np. Therefore, the axial extension Z_{FOV} of the surface enclosing the volume is covered by, i.e. equivalent to Np K, as can be deduced by inverting the previous formula, or by observing FIG. 2b.

As a result of the geometric magnification, the geometric footprint of the indentation on detector C has amplitude K′ which is given by the equation K′=K·(IDD+SFD+φ/2)/(SFD), where φ is the diameter of the FOV in the x-y plane. Therefore, the amplitude K is the axial extent of the beam footprint on the surface of the FOV facing the source and K′ is the axial extent of the beam footprint on the detector. The different footprints of axial amplitude K′ of the radiation x, on the detector, correspond to the footprints of axial amplitude K on the FOV.

SFD indicates the distance between source B and the closest point of the FOV surface.

To obtain contiguous scans along the z axis it is sufficient to consider the following relationship:

$$\frac{{Z}_{F O V}}{N p} = 2 \cdot SFD \cdot tan ( \frac{β}{2} )$$

The FOV according to a section in the plane orthogonal to the rotation axis D—x-y plane—has a circular shape with diameter φ and is the geometric footprint in the x-y plane of the points in space which are irradiated at least once for each cycle of oscillation of the source. The FOV has a maximum extension in the direction of the axis of rotation z equal to ZFOV.

For a fixed Z_{FOV} and a number of illuminations per cycle of oscillation during rotation equal to Np, the collimation must guarantee that the aperture of the x-ray beam in the z direction at a distance φ/2 of the axis D of rotation evaluated in the plane containing the source and axis of rotation is Z_{FOV}/Np. In other words, if the angular amplitude β of the beam on the rotation axis D and the axial amplitude Z_{FOV} of the FOV are fixed, then Np is obtained. Or if the number Np and the axial amplitude ZFOV of the FOV are fixed, then the angular amplitude β of the beam is obtained.

Axial positions Np of the source correspond to the Np footprints K and K′. The mutual distance between two consecutive axial positions of the source is denoted by FF. Instead, the source in the reciprocating axial motion sweeps a distance H equal to

$$H = FF \cdot ( N p - 1 )$$

Since the radiation has amplitude β it is evident that H is less than the length Z_{FOV}.

In FIG. 2bis it is foreseen to manipulate the opening angle in the extreme axial positions. In particular, the beam opening angle in the axial direction is halved at β/2.

This makes H coinciding with ZFOV.

During the rotation-oscillation motion, the source emits continuous or pulsed x-radiation. Contextually, the detector acquires the signal propagated through the sample by performing a scan of the sample.

Each of the footprints acquired per cycle of oscillation corresponds to a different position of the source identified by the coordinate in z and by the scanning angle σ between the x axis and the geometric footprint of the position of the source in the x-y plane. During the acquisition of the single projective image, the source can be fixed with respect to the sample for the duration of the single acquisition by performing a “step-and-shot” acquisition. In this case, the axial distance FF between two of the Np assumed by the source during irradiation is significant.

Alternatively, the scan may be continuous during the reciprocal motion between the sample and the source/detector pair. In both configurations, the x-ray source can operate in continuous x-ray emission mode or in pulsed x-ray emission mode, i.e. emit pulses of x-rays of fixed duration at a given period. In the first case the source emits photons x at every instant of the duration of the scan and a shutter device can be set up to temporarily limit the irradiation of the sample.

In the case of acquisition with the source in continuous movement, FF indicates the axial distance between two intermediate positions of the source corresponding to two consecutive footprints during its alternating movement parallel to the rotation axis D.

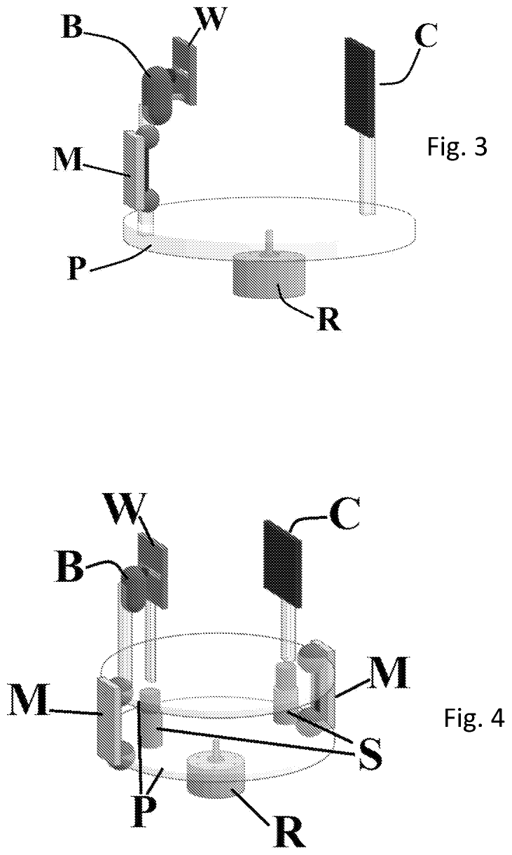

According to the present invention two variants can be envisaged:
a first variant provides, with reference to FIG. 3, that the detector is at least axially fixed, in the sense that it does not describe the alternating motion of the source. It has an extension in the axial direction such as to acquire the footprints of the x-ray beam, independently of the axial position of the source B which is instead driven by an axial motor M. The source B with the relative collimator W and the detector are fixed to a rotating platform P, around the rotation axis D perpendicular to the platform, for the engine R;
a second variant foresees, with reference to FIG. 4, that the detector C moves in accordance with the source B. Therefore, also the detector C is equipped with an axial motor M or is connected to the source B by means of a structure so that the motion along the D axis is simultaneous. Furthermore, the axial extension of the detector is limited such that it captures approximately the area of the beam footprint on the detector itself. According to the present invention, during the acquisition of the radiation by the detector, the scattering effect is reduced by cutting off the reflections which fall beyond the K′ footprint of the primary radiation x on the detector. According to the first variant, this cutting off is performed by digital image processing operations, i.e. by windowing the footprint of the primary radiation x on the detector, before recomposing the various footprints to obtain the three-dimensional reconstruction of the FOV. According to the second variant, this cutting off is constructive, in the sense that it may be obtained by sizing the detector so as to acquire only the axial impression K′. For this reason, the motion of the detector must be coordinated with the axial motion of the source.

Advantageously, this determines a reduction of the scatter radiation on the detector since the differential cross section of the Compton interaction—phenomenon at the basis of the formation of the scatter signal—in the energy ranges of photons x in medical CT applications has maximum values for angles greater than 10°. This allows to obtain a reconstruction of the FOV less affected by this contribution. This result is therefore obtained by refining the footprint on the detector or by reducing the axial extension of the detector to the footprint extension on it. Obviously, in both cases it is necessary to take into account the effect of beam collimation and the distances between the source and the detector and between the FOV and the detector. Therefore, according to the present invention, in order to remove the radiation signal outside the primary beam, i.e. what is outside the footprint having extension K′, the footprint is specially reduced (crop) through the removal of the pixels which are not in the primary field or the axial sizing of the detector such as to be strictly sufficient to capture the footprint K′ of the beam. According to the first variant, the windowing of the pixels generated by the detector is movable according to the axial position of the source. In the second variant, the entire detector is movable in accordance with the axial position of the source. Evidently, there must be only one radiation source x, otherwise the detector, inside a footprint, also receives the scatter signals of another footprint and the solution described here is ineffective. Multiple sources can also be used, but only one can be activated at a time. Therefore, the post-processing can be implemented through the knowledge of the reciprocal position source-detector during the axial movement, or by making the detector integral with the source also in the oscillation movements in the axial direction. If the detector and the source are integral in the movements, both rotary and translational in the axial direction, the position of the area irradiated by the primary beam remains fixed in the reference system integral with the detector; this area can be defined through a geometric calibration performed without a sample in the field of view, where the scatter radiation is limited and it is easy to detect the footprint of the primary beam on the detector. Conversely, when the detector is at least axially fixed, then a calibration can be performed, in the absence of the sample, by correlating the axial displacement of the source and the primary radiation signature on the detector.

It is worth highlighting that it is also possible to combine the two variants described above, in the sense that it is possible to have a detector with an axial dimension greater than K′ and also movable axially integrally with the source. In this case, post-processing of the digital image is necessary. This solution is advantageous because it allows to vary the distance between source and detector and the collimation width (B) to scan objects of different sizes.

It is therefore evident that the cutting of the image is made as a function of the distance between the Source and the detector and of the beam amplitude β.

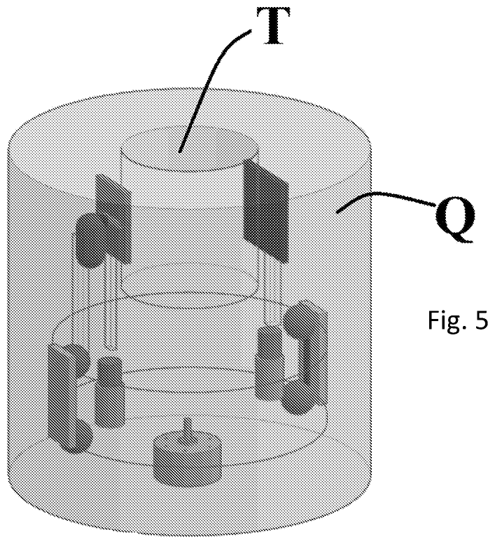

According to the variant of FIG. 5, a protective casing Q is provided to enclose the scanner and the relative sample during the acquisition procedure.

A special reconstruction software allows the reconstruction of the 3D map of the attenuation coefficients of the materials included in the FOV also allowing the evaluation of the values of the same in the Hounsfield scale, i.e. the evaluation of the value of the reconstructed voxel in Hounsfield Unit.

The present invention can advantageously be implemented through a computer program comprising coding means for carrying out one or more steps of the method, when this program is executed on a computer. Therefore, it is understood that the scope of protection extends to said computer program and also to computer-readable means comprising a recorded message, said computer-readable means comprising program coding means for carrying out one or more steps of the method, when said program is run on a computer.

Variants of the non-limiting example described are possible, without however departing from the scope of protection of the present invention, including all equivalent embodiments for a person skilled in the art, to the contents of the claims.

From the description given above, the person skilled in the art is capable of realizing the object of the invention without introducing further constructive details.

1. A CT computed tomography scanner comprising a source arranged to generate cone beam x-ray radiation, wherein the cone beam vertex is in the source defining a collimated cone, a detector arranged so that said radiation generates a primary footprint completely contained on a surface of the detector and processing means configured to generate a digital image corresponding to a signal generated by the detector when illuminated by said radiation and configured to reconstruct a map of three-dimensional attenuation of a volume containing a sample under investigation, wherein the source is arranged to create
a first rotating movement around an axis of rotation; and
a second reciprocating movement parallel to said axis of rotation so as to identify an approximately sinusoidal motion on a cylindrical or semi-cylindrical surface coaxial with said axis of rotation, having a predetermined amplitude,
and wherein the source is provided with a collimator arranged to illuminate only an axial fraction of a lateral surface enclosing said volume and wherein an entire axial extent of said lateral surface is irradiated over an entire period of oscillation comprising a predetermined number of footprints so that said axial extension is equal to the product of said axial fraction by said predetermined number of footprints, and wherein said detector has an axial extension approximately at least equal to an axial extension of said primary footprint.

2. The scanner according to claim 1, wherein the detector is fixed in the axial direction and has an axial extension at least equal to the primary footprint during said second reciprocating movement, so that said primary footprint is always contained in the surface of the detector, or wherein the detector is movable in said axial direction, synchronously and concordant with the source.

3. The scanner according to claim 1, wherein said processing means are configured to window said digital image in accordance with said primary footprint.

4. The scanner according to claim 1, wherein said source and said detector are adapted to rotate around said rotation axis in a manner integral with each other and wherein the detector can be axially fixed or axially mobile synchronously and in agreement with said source.

5. The scanner according to claim 1, wherein said alternating movement and said collimator are arranged so that said axial portions of the volume illuminated by the source during one oscillating period are contiguous and without overlapping for said entire oscillation period.

6. The scanner according to claim 1, wherein said source is associated with a turntable having a rotation axis coaxial with the rotation axis and wherein the source is associated with the platform by means of an axial actuator.

7. The scanner according to claim 5, wherein said detector is associated with said turntable and wherein said detector is associated with the turntable by means of said axial actuator or by means of a further axial actuator.

8. The scanner according to claim 1, wherein, considering a perpendicular plane with respect to said axis of rotation and passing through said detector, said detector is in a fixed angular position in said plane with respect to said rotation axis and said source tilts, in said plane, according to said first movement around said rotation axis for an angle lower than 180°.

9. A scanning method for making a CT computed tomography comprising a source arranged to generate cone beam x-ray radiation, wherein the cone beam vertex is in the source defining a collimated cone, a detector arranged in so that said radiation generates a primary footprint completely contained on a surface of the detector and processing means configured to generate a digital image corresponding to a signal generated by the detector when illuminated by said radiation and configured to reconstruct a three-dimensional attenuation map of a volume containing a sample under investigation, the method including
a first step of making said source perform a first tilting movement around an axis of rotation;
a second step of making said source perform a second alternating movement, parallel to said axis of rotation so as to identify an approximately sinusoidal motion on a cylindrical or hemicylindrical surface coaxial with said axis of rotation, having a predetermined amplitude;
a third step of collimating said beam so as to illuminate only an axial fraction of said volume;
a fourth step of performing two or more illuminations of the volume so that an entire axial extension of said lateral surface is irradiated through an entire period of oscillation comprising a predetermined number of footprints so that said axial extension is equal to the product of said axial fraction for said predetermined number of footprints; and
a fifth step of limiting said digital image by eliminating pixels outside said primary footprint.

10. The method according to claim 9, wherein said limitation is accomplished by an axial extension of the detector surface approximately equal to an axial extension of said primary footprint or by said processing means configured to window said digital image by eliminating pixels outside said primary footprint.
